(19) United States
(12) Patent Application Publication
(10) Pub. No.: US 2025/0116589 A1
(43) Pub. Date: Apr. 10, 2025

(54) SYSTEMS AND METHODS FOR QUANTIFYING AND MONITORING HYDROCARBON VOLUMES AND SURFACE GAS EMISSIONS FOR WIRELINE FORMATION TESTING

(71) Applicant: Schlumberger Technology Corporation, Sugar Land, TX (US)

(72) Inventors: Adriaan Gisolf, Voluntari (RO); Hua Yu, Beijing (CN); Yifei Wu, Sugar Land, TX (US); Richard Jackson, Houston, TX (US); Hadrien Dumont, Paris (FR); Youxiang Zuo, Edmonton (CA); Thomas Pfeiffer, Katy, TX (US); Kang Wang, Houston, TX (US); Simon Edmundson, Houston, TX (US)

(21) Appl. No.: 18/834,813

(22) PCT Filed: Mar. 27, 2023
(86) PCT No.: PCT/US2023/016362

§ 371 Date: Jul. 31, 2024

Related U.S. Application Data
(60) Provisional application No. 63/269,907, filed on Mar. 25, 2022.

(51) Int. Cl. G01N 9/32; E21B 49/08; G01N 33/28

(52) U.S. Cl. CPC G01N 9/32; E21B 49/0875; G01N 33/2823

(57) ABSTRACT

Systems and methods presented herein generally relate to a formation testing platform for quantifying and monitoring hydrocarbon volumes and surface gas emissions using formation testing data collected by a formation testing tool. For example, a method includes allowing one or more fluids from a subterranean formation to flow through a formation testing tool disposed in a wellbore of a well; determining, via the formation testing tool, data relating to one or more properties of the one or more fluids; communicating the data relating to the one or more properties of the one or more fluids from the formation testing tool to a surface control system; and determining, via the surface control system, hydrocarbon content of the one or more fluids and/or gas emissions relating to the one or more fluids based at least in part on the data relating to the one or more properties of the one or more fluids.

### CROSS-REFERENCE TO RELATED APPLICATIONS

This application claims benefit of U.S. Provisional Application No. 63/269,907 entitled “Systems and Methods for Quantifying and Monitoring Hydrocarbon Volumes and Surface Gas Emissions for Wireline Formation Testing,” filed Mar. 25, 2022, the disclosure of which is incorporated herein by reference in its entirety.

### BACKGROUND

The present disclosure generally relates to a formation testing platform for quantifying and monitoring hydrocarbon volumes and surface gas emissions using formation testing data collected by a formation testing tool.

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as an admission of any kind.

Field engineers running wireline formation testing operations routinely track the volume of fluid that is pumped into the wellbore. However, from a well control standpoint, the volumes of hydrocarbons and gas that are pumped into the wellbore mud column are often not quantified or accurately monitored. A common practice is to assume that the entire volume of fluids pumped into the wellbore is hydrocarbon or gas. This approach is not very reliable and can often lead to overestimates of the volume of produced hydrocarbon volumes, resulting in unnecessary and costly wiper trips to recondition and circulate the wellbore. Recent developments in formation testing and sampling technologies provide measurements to address this challenge and to enable quantification of hydrocarbon volumes and surface gas emissions. This is enabled by making use of available downhole fluid sensor and sampling measurements in substantially real-time to accurately quantify the amounts of hydrocarbons and gas that is being pumped into the wellbore during wireline formation transient testing and sampling operations.

Hydrocarbons and associated gas or free gas that is pumped into the wellbore during sampling and testing operations will eventually reach the surface. The hydrocarbons and gas are either immediately circulated to surface during deep transient testing (DTT) operations, or they are circulated to the surface during a subsequent wiper trip after wireline formation testing operations have been completed. Once at the surface, the methane and hydrocarbon gases may be separated out or, more typically, may evaporate and be vented to the atmosphere.

### SUMMARY

A summary of certain embodiments described herein is set forth below. It should be understood that these aspects are presented merely to provide the reader with a brief summary of these certain embodiments and that these aspects are not intended to limit the scope of this disclosure.

Certain embodiments of the present disclosure include a method that includes allowing one or more fluids from a subterranean formation to flow through a formation testing tool disposed in a wellbore of a well. The method also includes determining, via the formation testing tool, data relating to one or more properties of the one or more fluids. The method further includes communicating the data relating to the one or more properties of the one or more fluids from the formation testing tool to a surface control system. In addition, the method includes determining, via the surface control system, hydrocarbon content of the one or more fluids and/or gas emissions relating to the one or more fluids based at least in part on the data relating to the one or more properties of the one or more fluids.

Certain embodiments of the present disclosure also include a system that includes a formation testing tool configured to receive one or more fluids from a subterranean formation while the formation testing tool is disposed in a wellbore of a well, and to determine data relating to one or more properties of the one or more fluids. The system also includes a surface control system configured to receive the data relating to the one or more properties of the one or more fluids from the formation testing tool, and to determine hydrocarbon content of the one or more fluids and/or gas emissions relating to the one or more fluids based at least in part on the data relating to the one or more properties of the one or more fluids.

Certain embodiments of the present disclosure also include a formation testing platform configured to receive one or more fluids from a subterranean formation while a formation testing tool is disposed in a wellbore of a well, to determine data relating to one or more properties of the one or more fluids, and to determine hydrocarbon content of the one or more fluids and/or gas emissions relating to the one or more fluids based at least in part on the data relating to the one or more properties of the one or more fluids.

Various refinements of the features noted above may be undertaken in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended to familiarize the reader with certain aspects and contexts of embodiments of the present disclosure without limitation to the claimed subject matter.

### BRIEF DESCRIPTION OF THE DRAWINGS

Various aspects of this disclosure may be better understood upon reading the following detailed description and upon reference to the drawings, in which:

FIG. 1 is a schematic diagram of a formation testing tool, in accordance with embodiments of the present disclosure;

FIG. 2 illustrates a surface control system that may control the oil and gas well system of FIG. 1, in accordance with embodiments of the present disclosure;

FIG. 3 is a side view of a formation testing tool, in accordance with embodiments of the present disclosure;

FIG. 4 illustrates an embodiment of a tool control system illustrated in FIG. 1, in accordance with embodiments of the present disclosure;

FIG. 5 illustrates a formation testing tool with a dual flowline, in accordance with embodiments of the present disclosure;

FIG. 6 is a flow diagram of a workflow that may be utilized by the formation testing tool to determine the total mass rate of the fluid flowing through the formation testing tool, in accordance with embodiments of the present disclosure;

FIG. 7 is a flow diagram of a workflow that may be utilized by the formation testing tool to determine the total mass rate of gas, in accordance with embodiments of the present disclosure; and

FIG. 8 is a flow diagram of a workflow that may be utilized by the formation testing tool to determine the total pumped gas volume accumulated at the surface at standard conditions for each component from the various fluids/flowlines, in accordance with embodiments of the present disclosure.

### DETAILED DESCRIPTION

One or more specific embodiments of the present disclosure will be described below. These described embodiments are only examples of the presently disclosed techniques. Additionally, in an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

As used herein, the terms “connect,” “connection,” “connected,” “in connection with,” and “connecting” are used to mean “in direct connection with” or “in connection with via one or more elements”; and the term “set” is used to mean “one element” or “more than one element.” Further, the terms “couple,” “coupling,” “coupled,” “coupled together,” and “coupled with” are used to mean “directly coupled together” or “coupled together via one or more elements.”

In addition, as used herein, the terms “real time”, “real-time”, or “substantially real time” may be used interchangeably and are intended to describe operations (e.g., computing operations) that are performed without any human-perceivable interruption between operations. For example, as used herein, data relating to the systems described herein may be collected, transmitted, and/or used in control computations in “substantially real time” such that data readings, data transfers, and/or data processing steps occur once every second, once every 0.1 second, once every 0.01 second, or even more frequently, during operations of the systems (e.g., while the systems are operating). In addition, as used herein, the terms “continuous”, “continuously”, or “continually” are intended to describe operations that are performed without any significant interruption. For example, as used herein, control commands may be transmitted to certain equipment every five minutes, every minute, every 30 seconds, every 15 seconds, every 10 seconds, every 5 seconds, or even more often, such that operating parameters of the equipment may be adjusted without any significant interruption to the closed-loop control of the equipment. In addition, as used herein, the terms “automatic”, “automated”, “autonomous”, and so forth, are intended to describe operations that are performed are caused to be performed, for example, by a computing system (i.e., solely by the computing system, without human intervention).

The formation testing platform described herein provides measurements of pressure, temperature, volumetric flowrate, and total flowed volume, among other operational parameters, versus elapsed time. In addition, the embodiments described herein include downhole fluid analysis (DFA) sensors to measure and determine fluid properties such as hydrocarbon composition (e.g., weight fractions of CO₂, C₁, C₂, C₃, C₄, C₅, C₆⁺, and so forth), fluid density, mud filtrate contamination level, gas/oil ratio (GOR), and formation volume factors, among other properties, during a test station. Through integration of the DFA sensor measurements with the station transient data such as flowrate, mass and molar flowrates may be derived for each component, and the total mass and mole of gas pumped from the formation, in substantially real time every time fluid is pumped out from the formation into the wellbore. The mass rate of the individual components can then be converted to surface rates and surface volumes, thereby enabling accurate determination and monitoring of surface gas emissions resulting from any downhole fluid pumped during formation testing and sampling operations. In addition, the embodiments described herein include a workflow to enable effective monitoring and control of surface gas emissions during formation testing operations. The ability to quantify and monitor surface emissions is also an important first step to help enable reductions in CO₂ and greenhouse gas emissions, which also aligns with global sustainable development goals.

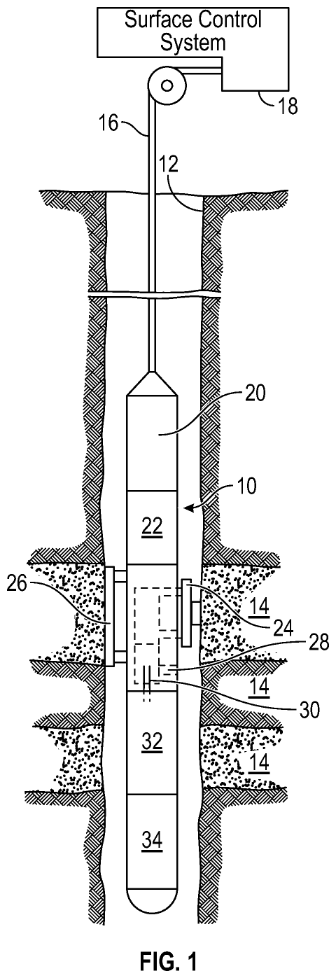

FIG. 1 illustrates a formation testing tool 10. As illustrated, in certain embodiments, the formation testing tool 10 may be suspended in a wellbore 12 traversing a formation 14 by a cable 16 (e.g., a wireline cable) that is spooled in a usual fashion on a suitable winch (not shown) on the formation surface. On the surface, the cable 16 may be electrically coupled to a surface control system 18. As illustrated, in certain embodiments, the formation testing tool 10 includes an elongated body 20 that encloses a tool control system 22. In certain embodiments, the elongated body 20 also includes a fluid admitting assembly 24 and a tool anchoring member 26, which may be arranged on opposite lateral sides of the body 20. In certain embodiments, the fluid admitting assembly 24 is configured to selectively seal off or isolate selected portions of the wall of the wellbore 12 such that pressure or fluid communication with the adjacent formation 14 is established. In addition, in certain embodiments, the formation testing tool 10 includes a fluid analysis module 28 with a flow line 30 through which fluid collected from the formation 14 flows. The fluid may thereafter be expelled through a port (not shown) or may be directed to one or more fluid collecting chambers 32, 34, which may receive and retain the fluids collected from the formation 14. As described in greater detail herein, the fluid admitting assembly 24, the fluid analysis module 28, and the flow path to the fluid collecting chambers 32, 34 may be controlled by the control systems 18, 22.

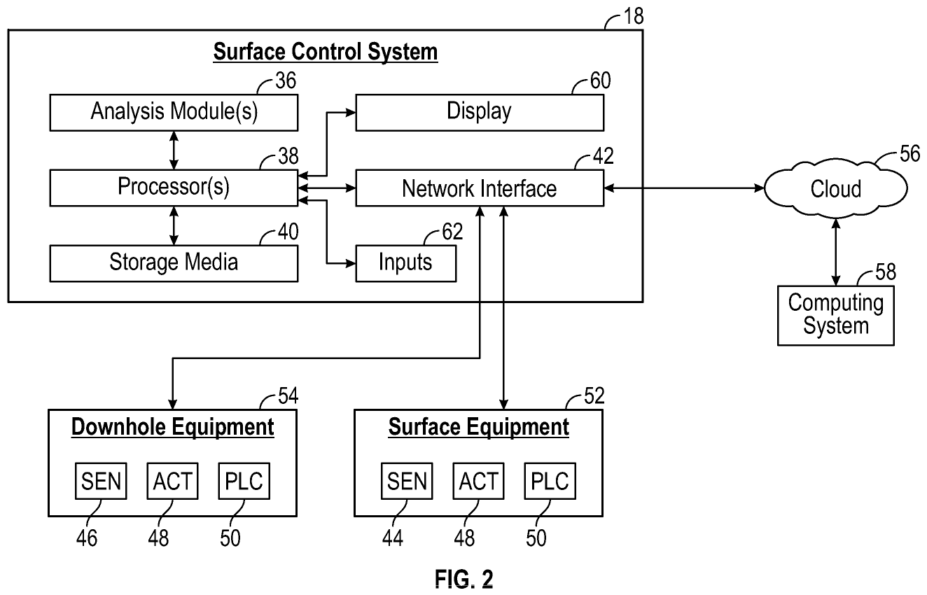

FIG. 2 illustrates an embodiment of the surface control system 18 illustrated in FIG. 1. In certain embodiments, the surface control system 18 may include one or more analysis modules 36 (e.g., a program of processor executable instructions and associated data) that may be configured to perform various functions of the embodiments described herein. In certain embodiments, to perform these various functions, an analysis module 36 executes on one or more processors 38 of the surface control system 18, which may be connected to one or more storage media 40 of the surface control system 18. Indeed, in certain embodiments, the one or more analysis modules 36 may be stored in the one or more storage media 40.

In certain embodiments, the one or more processors 38 may include a microprocessor, a microcontroller, a processor module or subsystem, a programmable integrated circuit, a programmable gate array, a digital signal processor (DSP), or another control or computing device. In certain embodiments, the one or more storage media 40 may be implemented as one or more non-transitory computer-readable or machine-readable storage media. In addition, in certain embodiments, the one or more storage media 40 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories; magnetic disks such as fixed, floppy and removable disks; other magnetic media including tape; optical media such as compact disks (CDs) or digital video disks (DVDs); or other types of storage devices. Note that the processor-executable instructions and associated data of the analysis module(s) 36 may be provided on one computer-readable or machine-readable storage medium of the storage media 40, or alternatively, may be provided on multiple computer-readable or machine-readable storage media distributed in a large system having possibly plural nodes. Such computer-readable or machine-readable storage medium or media are considered to be part of an article (or article of manufacture), which may refer to any manufactured single component or multiple components. In certain embodiments, the one or more storage media 40 may be located either in the machine running the machine-readable instructions, or may be located at a remote site from which machine-readable instructions may be downloaded over a network for execution.

In certain embodiments, the processor(s) 38 may be connected to a network interface 42 of the surface control system 18 to allow the surface control system 18 to communicate with various surface sensors 44 and/or downhole sensors 46 described herein, as well as communicate with various actuators 48 and/or PLCs 50 of surface equipment 52 (e.g., surface pumps, valves, and so forth) and/or of downhole equipment 54 (e.g., the formation testing tool 10, electric submersible pumps, other downhole tools, and so forth) for the purpose of controlling operation of the oil and gas well system illustrated in FIG. 1. In certain embodiments, the network interface 42 may also facilitate the surface control system 18 to communicate data to a cloud-based service 56 (or other wired and/or wireless communication network) to, for example, archive the data or to enable external computing systems 58 (e.g., cloud-based computing systems, in certain embodiments) to access the data and/or to remotely interact with the surface control system 18. For example, in certain embodiments, some or all of the analysis modules 36 described in greater detail herein may be executed via cloud and edge deployments.

In certain embodiments, the surface control system 18 may include a display 60 configured to display a graphical user interface to present results on the control of the formation testing operations described herein. In addition, in certain embodiments, the graphical user interface may present other information to operators of the equipment 52, 54 described herein. For example, the graphical user interface may include a dashboard configured to present visual information to the operators. In certain embodiments, the dashboard may show live (e.g., real-time) data as well as the results of the control of the formation testing operations described herein.

In addition, in certain embodiments, the surface control system 18 may include one or more input devices 62 configured to enable operators to, for example, provide commands to the equipment 52, 54 described herein. For example, in certain embodiments, the formation testing tool 10 may provide information to the operators regarding the formation testing operations, and the operators may implement actions relating to the formation testing operations by manipulating the one or more input devices 62, as described in greater detail herein. In certain embodiments, the display 60 may include a touch screen interface configured to receive inputs from operators. For example, an operator may directly provide instructions to the formation testing tool 10 via the user interface, and the instructions may be output to the formation testing tool 10 via a controller and a communication system of the formation testing tool 10.

It should be appreciated that the surface control system 18 illustrated in FIG. 2 is only one example of a well control system, and that the surface control system 18 may have more or fewer components than shown, may combine additional components not depicted in the embodiment of FIG. 2, and/or the surface control system 18 may have a different configuration or arrangement of the components depicted in FIG. 2. In addition, the various components illustrated in FIG. 2 may be implemented in hardware, software, or a combination of both hardware and software, including one or more signal processing and/or application specific integrated circuits. Furthermore, the operations of the surface control system 18 as described herein may be implemented by running one or more functional modules in an information processing apparatus such as application specific chips, such as application-specific integrated circuits (ASICs), field-programmable gate arrays (FPGAs), programmable logic devices (PLDs), systems on a chip (SOCs), or other appropriate devices. These modules, combinations of these modules, and/or their combination with hardware are all included within the scope of the embodiments described herein.

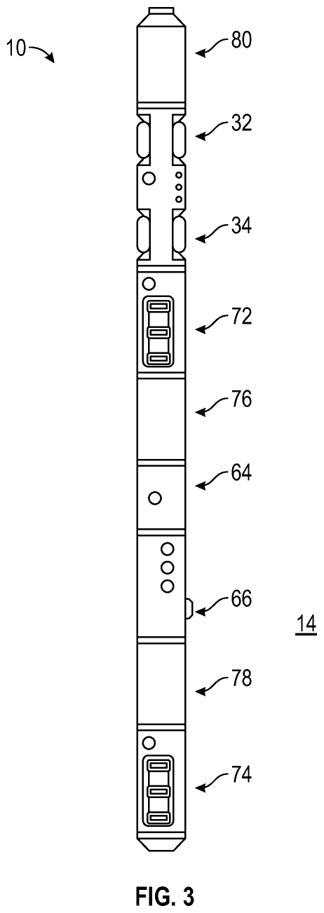

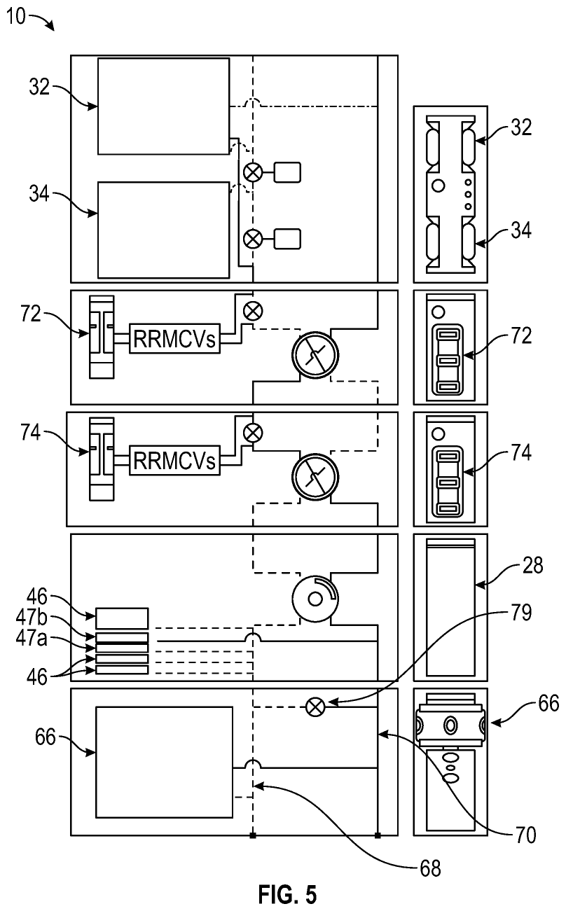

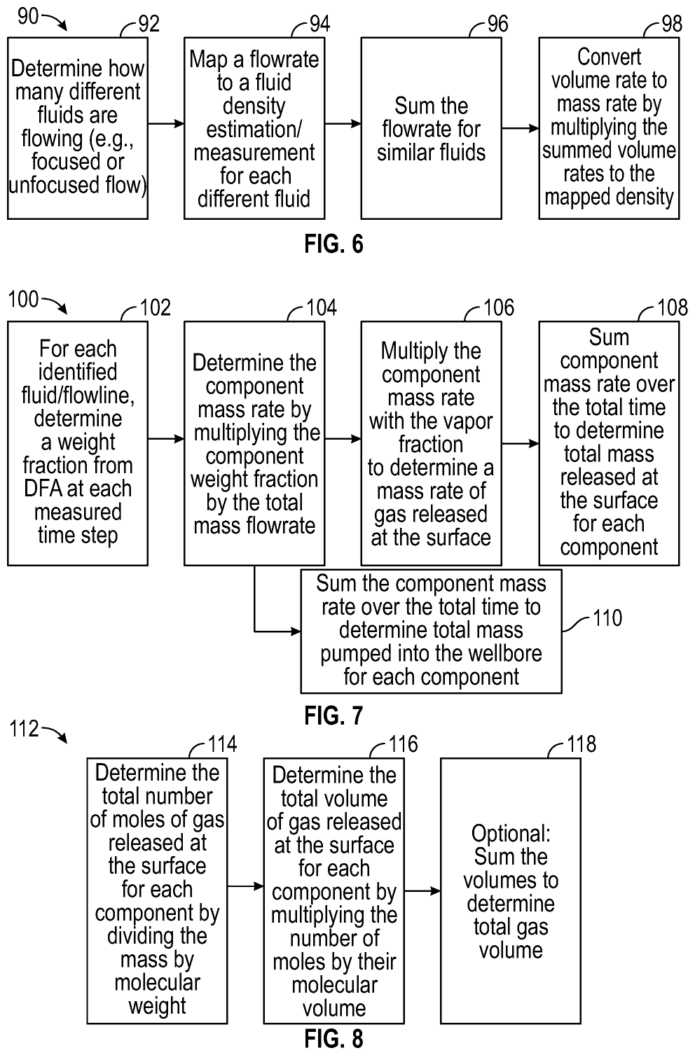

As described above, the embodiments described herein include a formation testing tool 10 configured to perform reservoir fluid analysis by drawing in formation fluid and testing the formation fluid downhole or collecting a sample of the formation fluid to bring to the surface. For example, in certain embodiments, the formation testing tool 10 may use a probe and/or packers to isolate a desired region of the wellbore 12 (e.g., at a desired depth) and establish fluid communication with the subterranean formation 14 surrounding the wellbore 12. The probe may draw the formation fluid into the formation testing tool 10. For example, FIG. 3 is a side view of a formation testing tool 10. As illustrated in FIG. 3, the formation testing tool 10 may include a hydraulic module 64 configured to control the flow of fluid through fluid lines of the formation testing tool 10, and a probe 66 that includes one or more inlets for receiving the fluid through the fluid lines of the formation testing tool 10. In certain embodiments, the probe 66 may include multiple inlets (e.g., a sampling probe and a guard probe) that may be used for the sampling described herein. In such embodiments, the probe 66 may be connected to sampling flowlines (not explicitly shown in FIG. 3; see e.g., 68 in FIGS. 5 and 6), as well as to guard flowlines (not explicitly shown in FIG. 3; see e.g., 70 in FIGS. 5 and 6). In certain embodiments, the probe 66 may be movable between extended and retracted positions for selectively engaging the wellbore 12 and acquiring fluid samples from the formation 14. In addition, in certain embodiments, a sampling pump module 72 and a guard pump module 74 configured to aid the flow of the fluid through the respective flowlines 68, 70.

As described in greater detail herein, the formation testing tool 10 also includes a fluid analysis module 28 configured to analyze the fluid flowing through the sampling flowline 68 and the guard flowline 70. In particular, the fluid analysis module 28 may include a sampling fluid analyzer 76 and a guard fluid analyzer 78 configured to analyze the fluid flowing through the respective flowlines 68, 70. In addition, as described above, the formation testing tool 10 includes one or more fluid collecting chambers 32, 34 configured to store the fluid samples. In addition, in certain embodiments, the formation testing tool 10 may include a power cartridge 80 configured to receive electrical power from the cable 16 and supply suitable voltages to the electronic components of the formation testing tool 10.

In addition, as described above, the formation testing tool 10 includes a tool control system 22 (not explicitly shown in FIG. 3) that controls the local functionality of the formation testing tool 10. In certain embodiments, the tool control system 22 of the formation testing tool 10 may communicate with the surface control system 18 such that the control systems 18, 22 collectively control operation of the formation testing tool 10. As will be appreciated, the tool control system 22 of the formation testing tool 10 may include components that are substantially similar to the components of the surface control system 18 illustrated in FIG. 2, other than the display 60 and the input devices 62.

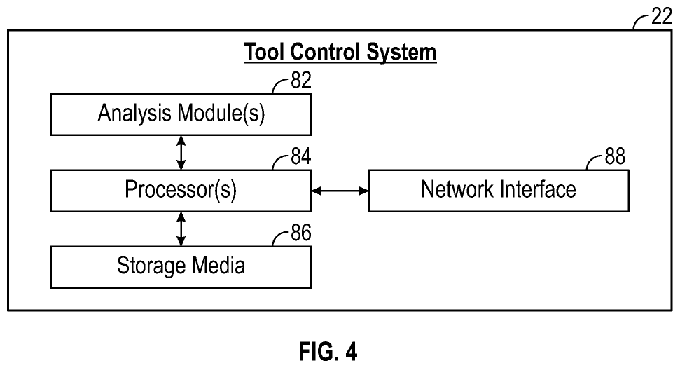

FIG. 4 illustrates an embodiment of the tool control system 22 illustrated in FIG. 1. In certain embodiments, the tool control system 22 may include one or more analysis modules 82 (e.g., a program of processor executable instructions and associated data) that may be configured to perform various functions of the embodiments described herein. In certain embodiments, to perform these various functions, an analysis module 82 executes on one or more processors 84 of the tool control system 22, which may be connected to one or more storage media 86 of the tool control system 22. Indeed, in certain embodiments, the one or more analysis modules 82 may be stored in the one or more storage media 86.

In certain embodiments, the one or more processors 84 may include a microprocessor, a microcontroller, a processor module or subsystem, a programmable integrated circuit, a programmable gate array, a digital signal processor (DSP), or another control or computing device. In certain embodiments, the one or more storage media 86 may be implemented as one or more non-transitory computer-readable or machine-readable storage media. In addition, in certain embodiments, the one or more storage media 86 may include one or more different forms of memory including semiconductor memory devices such as dynamic or static random access memories (DRAMs or SRAMs), erasable and programmable read-only memories (EPROMs), electrically erasable and programmable read-only memories (EEPROMs) and flash memories; or other types of storage devices. Such computer-readable or machine-readable storage medium or media are considered to be part of an article (or article of manufacture), which may refer to any manufactured single component or multiple components. In addition, in certain embodiments, the processor(s) 84 may be connected to a network interface 88 of the tool control system 22 to allow the tool control system 22 to communicate with the surface control system 18.

As described in greater detail herein, the formation testing platform described herein performs various specific analysis including, but not limited to, (1) determining the total pumped volume of fluid flowing through the formation testing tool 10, (2) determining the mass of gas of the individual components of the fluid flowing through the formation testing tool 10, and (3) determining the volume of individual gas components, (equivalent) greenhouse gas, and total volume of gas at the surface.

When fluid is pumped from a formation 14 into the formation testing tool 10, the fluid is moved from an inlet of the formation testing tool 10, through the sample flowline 68 and the guard flowline 70, and either deposited in the wellbore 12 or circulated to the surface. Volumetric flowrate and live fluid density of the fluid may be measured by the formation testing tool 10, along with the flowing temperature and pressure. In one or more embodiments, the volumetric flowrate may be determined by counting a number of turns of the motor of a known volume. Further, in one or more embodiments, the fluid density of the fluid within the sample flowline 68 may be measured by a first density sensor 47a and the fluid density of the fluid within the guard flowline 70 may be measured by a second density sensor 47b. In addition, the volumetric flowrate and the fluid density may be calculated by the formation testing tool 10 separately for each of the sampling flowline 68 and the guard flowline 70 when the fluid is different within each flowline 68, 70.

The formation testing tool 10 may be operated in various different modes. For example, in certain embodiments, the formation testing tool 10 may be operated in a focused mode with two pump modules 72, 74 on a single flowline toolstring (e.g., FIG. 3). In one or more embodiments, a focused mode is one in which the formation fluid within each of the sample flowline and the guard flowline of the formation testing tool 10 is not comingled, and thus is different. In this mode, the flowrate for each pump module 72, 74 may be determined by the formation testing tool 10 and the density of the fluid in each flowline may be determined by a density sensor disposed on said flowline (not explicitly shown in FIG. 3) so that both the volumetric rate and density are measured. In addition, in certain embodiments, the formation testing tool 10 may be operated in a focused mode with a dual flowline tool (e.g., FIG. 5). In one or more embodiments, the dual flowline tool may be in a focused mode by maintaining a valve 79 between the sample flowline 68 and the guard flowline 70 closed. In this mode, the flowrate for each pump module 72, 74 may also be determined by the formation testing tool 10 and the density of the fluid in each flowline may be determined by a density sensor 47a, 47b disposed along said flowline so that both the volumetric rate and density are measured. In addition, in certain embodiments, the formation testing tool 10 may be operated in an unfocused mode with a dual flowline tool (e.g., FIG. 5). In one or more embodiments, the dual flowline tool may be in an unfocused mode by maintaining the valve 79 between the sample flowline 68 and the guard flowline 70 open, thus comingling the formation fluid in the two flowlines. In this mode, the flowrate may be calculated by the formation testing tool 10 as a summation of all flowrates from all active pumps. In certain embodiments operated in an unfocused mode, the density of the fluid may be determined by one or both of the density sensors 47a, 47b. In addition, in certain embodiments, the formation testing tool 10 may be operated in an unfocused mode with a single flowline tool. In this mode, the flowrate may also be calculated by the formation testing tool 10 as a summation of all flowrates from all active pumps. In certain embodiments operated in an unfocused mode, the density of the fluid may be determined by one or both of the density sensors disposed along the sample flowline or the guard flowline. While a valve 79 is depicted for separating or allowing comingling of formation fluid in the sampling flowline 68 and the guard flowline 70, in one or more embodiments, any suitable means may be employed that allow for either separating or comingling the formation fluid within each flowline.

FIG. 6 is a flow diagram of a workflow 90 that may be utilized by the formation testing tool 10 to determine the total mass rate of the fluid flowing through the formation testing tool 10. As illustrated, in certain embodiments, the workflow 90 may include determining how many different fluids (e.g., 1, 2, or even more) are flowing (e.g., either focused or unfocused flow) through the formation testing tool 10 (block 92). In addition, in certain embodiments, the workflow 90 may include mapping a flowrate to a fluid density estimation/measurement for each different fluid (block 94). In addition, in certain embodiments, the workflow 90 may include summing the flowrates for similar fluids (block 96). In addition, in certain embodiments, the workflow 90 may include converting volume rate to mass rate by multiplying the summed volume rates to the mapped density (block 98). It should be noted that fluid density may either be directly measured with a fluid density sensor of the formation testing tool 10, or estimated by the formation testing tool 10 using fluid compositional measurements and a fluid model. In certain embodiments, each of the steps of the workflow 90 may be performed by the formation testing tool 10.

The gas mass for each component in a pumped mixture (of hydrocarbon and filtrate) is of particular importance in dynamic well control (e.g., predicting the interaction of pumped fluids with the mud in the wellbore 12) and to track the mass of pumped gas and surface gas emissions. By computing this quantitative indicator in substantially real time, a pumped gas log may be generated for real-time monitoring and control.

For wells containing oil-based mud (OBM), the hydrocarbon will dissolve in the wellbore mud. When circulated to the surface, most of the gaseous components will come out of solution, and may be vented to the atmosphere as free gas (or potentially flared or otherwise treated). The fraction of gas that comes out of solution at the surface is called the vapor fraction. The vapor fractions of CO₂, C₁, C₂, C₃, C₄ and C₅ depends on the type of oil, while C₆⁺ is mainly a liquid component. The vapor fractions may be estimated in many different ways, including empirical methods, correlations, or by using a convolutional neural network (CNN) model, a recurrent neural network (RNN) model, or an artificial neural network (ANN) model, or other model. In certain embodiments, the input to determine the vapor fractions may be based on the measured fluid GOR, density, other measured fluid properties, and potentially mud-type and circulation rate. Alternatively, some vapor fractions may be set to 1 for certain mud/hydrocarbon combinations. For example, in the case of wells containing water-based mud (WBM) the CO₂, C₁, and C₂ vapor fractions might be set to 1. Note that in certain environments, it may be preferred to estimate an upper limit of the pumped gas rather than taking the risk of underestimation. Therefore, under certain scenarios, the vapor fractions of C₃-C₅ may be regarded as 1 if there is no better estimation.

FIG. 7 is a flow diagram of a workflow 100 that may be utilized by the formation testing tool 10 to determine the total mass rate of gas. As illustrated, in certain embodiments, the workflow 100 may include, for each fluid/flowline identified in the workflow 90 illustrated in FIG. 6, determining a weight fraction from DFA at each measured time step (block 102). In addition, in certain embodiments, the workflow 100 may include determining the mass rate of each component of the fluid by multiplying the weight fraction of each component by the total mass flowrate (block 104). In addition, in certain embodiments, the workflow 100 may include multiplying the component mass rate calculated in block 104 with the vapor fraction to determine a mass rate of gas released at the surface (block 106). In addition, in certain embodiments, the workflow 100 may include summing the component mass rate over the total time to determine total mass of gas released at the surface for each component (block 108). In addition, in certain embodiments, the workflow 100 may also include summing the component mass rate calculated in block 104 over the total time to determine total mass pumped into the wellbore 12 for each component (block 110). In certain embodiments, some of the steps of the workflow 100 may be performed by the formation testing tool 10 while other steps of the workflow 100 may be performed by the surface control system 18.

FIG. 8 is a flow diagram of a workflow 112 that may be utilized by the formation testing tool 10 to determine the total pumped gas volume accumulated at the surface at standard conditions for each component from the various fluids/flowlines. As illustrated, in certain embodiments, the workflow 112 may include determining the total number of moles of gas released at the surface for each component by dividing the mass by molecular weight (block 114). In addition, in certain embodiments, the workflow 112 may include determining the total volume of gas released at the surface for each component by multiplying the number of moles by their respective molecular volume (block 116). In addition, in certain embodiments, the workflow 112 may optionally include summing the volumes to determine total gas volume (block 118). In certain embodiments, each of the steps of the workflow 112 may be performed by the surface control system 18. Table 1 illustrates molar weights of each gas component (MW)).

TABLE 1
Molar weights of each gas component.

| Component | Molar Weight (g/mole) |
|---|---|
| Methane | 16.04 |
| Ethane | 30.07 |
| Propane | 44.10 |
| Butane | 58.12 |
| Pentane | 72.15 |
| CO₂ | 44.01 |

Alternatively, in certain embodiments, the individual component mass rates determined in the workflow 100 may be determined by dividing the mass of each component by total molecular weight first to determine the molecular rate, which may then be multiplied by the total molecular volume to determine the individual volume rates of each component. These can be summed over time to determine the individual component total volume at the surface.

In certain embodiments, determining the actual gas emission rates at the surface at standard conditions during DTT operations includes combining the wellbore volume and mud circulation rates. The mass rate and volume rates predicted in workflows 110, 112 may arrive at the surface, delayed by the circulation time, which is the wellbore volume divided by the mud circulation rates.

In certain embodiments, both methane and carbon dioxide are considered “greenhouse gas”. The mass and volume of these gases may be measured directly using the techniques described herein. Methane is much more potent than carbon dioxide when it comes to trapping heat in the atmosphere. It is, therefore, relatively important to be able to quantify the CO₂ equivalent effect of CH₄ and the CO₂ emissions in case CH₄ is flared. However, it is important to note measurements of CH₄ and CO₂ emissions are needed to be able to apply these conversions.

In certain embodiments, the summing of the total gas may be performed in an alternative manner by, for example, giving extra weight to heavier fractions using weighting factors. As but one non-limiting example, the total gas could be calculated using the equation:

$$Total gas = 1 \times {C}_{1} + 2 \times {C}_{2} + 3 \times {C}_{3} + 4 \times {C}_{4} + 5 \times {C}_{5} + 6 \times {C}_{6}$$

The information determined by the workflows 90, 100, 112 illustrated in FIGS. 7-9 may be used in various specific applications. As but one non-limiting example, in certain embodiments, the hydrocarbon footprint of any formation testing operation may be quantified. An important step to do so includes quantifying the individual gas component volumes and the total CO₂ equivalent volume released to the atmosphere due to pumping fluids from the formation 14 with the formation testing tool 10.

As another example, in certain embodiments, the reduction of emissions compared to other technologies may be quantified. The ability to quantify the emissions of each method/technology is a relatively important first step in reducing the total emissions. During DTT operations, the volume pumped to generate the pressure-transient build up is considerably larger than during wireline formation testing operations, but orders of magnitude smaller than during drill string testing (DST) operations. However, during a DST the produced HC are typically flared. The reduced volumes during a DTT (compared to a DST) result in less produced hydrocarbon at the surface. However, without quantifying the actual volume of gas released at the surface, it may be relatively difficult to do a quantitively emission comparison between different services, to quantify the emission effects of DTT design changes, such as changing the flowrate, flow duration, or the number of stations, and to quantify the emission effects of changing mud type and circulation rates.

As another example, in certain embodiments, before a formation testing operation, the pressure and formation fluid volume pumping limits may be simulated in advance. Doing so may serve as the limiting factor in the amount of hydrocarbons allowed to pump into the well. During the formation testing operations, the formation testing tool 10 may pump fluids continuously, and the volume of pumped hydrocarbons or gas may either fully or partially dissolve in OBM or be suspended in the wellbore 12 in WBM environments. In both cases a plume of gas-cut mud may tend to initiate and then accumulate downhole in the wellbore 12 near the test interval depths. These hydrocarbon plumes tend to remain downhole until they are circulated out. In certain embodiments, the formation testing tool 10 may measure the flowrate, composition, density, water fraction, and so forth, in substantially real time, and the methods described herein may be used to accurately estimate the total mass of gas. Formation testing operations may continue until the total gas mass limit is reached. The current limits are typically set based on total volume pumped rather than the mass of the gaseous components. It should be noted that because the amount of gas may be accurately estimated rather than relying on overly conservative limits (which is the current practice), the methods described herein allow unnecessary wiper trips to be prevented and mitigate potential well control risks.

In certain embodiments, after the formation testing run, drill pipe is lowered into the well. The gas-cut plume of mud may be pushed upwards by the displacement of the drill string. The amount of gas-cut mud needs to be limited, so that the plume stays below a safe depth and below the blowout preventer (BOP). When the drill pipe reaches target depth (TD), the plume may be circulated out of the hole.

The specific embodiments described above have been illustrated by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A method, comprising:
allowing one or more fluids from a subterranean formation to flow through a formation testing tool disposed in a wellbore of a well;
determining, via the formation testing tool, data relating to one or more properties of the one or more fluids;
communicating the data relating to the one or more properties of the one or more fluids from the formation testing tool to a surface control system; and
determining, via the surface control system, hydrocarbon content of the one or more fluids and/or gas emissions relating to the one or more fluids based at least in part on the data relating to the one or more properties of the one or more fluids.

2. The method of claim 1, comprising:
determining, via the formation testing tool, how many different fluids are flowing through the formation testing tool;
mapping, via the formation testing tool, a flowrate to a fluid density measurement for each fluid of the one or more fluids;
summing, via the formation testing tool, flowrates of similar fluids of the one or more fluids; and
converting, via the formation testing tool, the summed flowrates from volume rates to mass rates by multiplying the summed volume rates to the mapped fluid density measurement.

3. The method of claim 2, wherein the fluid density measurement comprises a direct measurement of the fluid density via a fluid density sensor of the formation testing tool.

4. The method of claim 2, wherein the fluid density measurement comprises an estimate of the fluid density performed by the formation testing tool using one or more compositional measurements of the one or more fluids and a fluid model.

5. The method of claim 1, comprising:
determining, via the formation testing tool, a weight fraction for each fluid of the one or more fluids at a plurality of time steps; and
determining, via the formation testing tool, a mass rate of each component of each fluid of the one or more fluids by multiplying the weight fraction for each component of each fluid of the one or more fluids by a total mass flowrate of the one or more fluids.

6. The method of claim 5, comprising:
determining, via the surface control system, a mass rate of gas released at a surface of the well by multiplying the mass rate of each component of each fluid of the one or more fluids with a vapor fraction of each component of each fluid of the one or more fluids; and
determining, via the surface control system, a total mass of gas released at the surface of the well for each component of each fluid of the one or more fluids by summing the mass rate of gas released at the surface of the well over total time of the plurality of time steps.

7. The method of claim 5, comprising determining, via the surface control system, a total mass pumped into the wellbore of the well for each component of each fluid of the one or more fluids by summing the mass rate of each component of each fluid of the one or more fluids over total time of the plurality of time steps.

8. The method of claim 1, comprising:
determining, via the surface control system, a total number of moles of gas released at a surface of the well for each component of each fluid of the one or more fluids by dividing the mass of each component of each fluid of the one or more fluids by molecular weight of the one or more fluids; and
determining, via the surface control system, a total volume of gas released at the surface of the well for each component of each fluid of the one or more fluids by multiplying a number of moles for each component of each fluid of the one or more fluids by a molecular volume for each component of each fluid of the one or more fluids.

9. The method of claim 8, comprising determining, via the surface control system, a total volume of gas released at the surface of the well by summing the total volume of gas released at the surface of the well for each component of each fluid of the one or more fluids.

10. A system, comprising:
a formation testing tool configured to receive one or more fluids from a subterranean formation while the formation testing tool is disposed in a wellbore of a well, and to determine data relating to one or more properties of the one or more fluids; and
a surface control system configured to receive the data relating to the one or more properties of the one or more fluids from the formation testing tool, and to determine hydrocarbon content of the one or more fluids and/or gas emissions relating to the one or more fluids based at least in part on the data relating to the one or more properties of the one or more fluids.

11. The system of claim 10, wherein the formation testing tool is configured to:
determine how many different fluids are flowing through the formation testing tool;
map a flowrate to a fluid density measurement for each fluid of the one or more fluids;
sum flowrates of similar fluids of the one or more fluids; and
convert the summed flowrates from volume rates to mass rates by multiplying the summed volume rates to the mapped fluid density measurement.

12. The system of claim 11, wherein the fluid density measurement comprises a direct measurement of the fluid density via a fluid density sensor of the formation testing tool.

13. The system of claim 11, wherein the fluid density measurement comprises an estimate of the fluid density performed by the formation testing tool using one or more compositional measurements of the one or more fluids and a fluid model.

14. The system of claim 10, wherein the formation testing tool is configured to:
determine a weight fraction for each fluid of the one or more fluids at a plurality of time steps; and
determine a mass rate of each component of each fluid of the one or more fluids by multiplying the weight fraction for each component of each fluid of the one or more fluids by a total mass flowrate of the one or more fluids.

15. The system of claim 14, wherein the surface control system is configured to:
determine a mass rate of gas released at a surface of the well by multiplying the mass rate of each component of each fluid of the one or more fluids with a vapor fraction of each component of each fluid of the one or more fluids; and
determine a total mass of gas released at the surface of the well for each component of each fluid of the one or more fluids by summing the mass rate of gas released at the surface of the well over total time of the plurality of time steps.

16. The system of claim 14, wherein the surface control system is configured to determine a total mass pumped into the wellbore of the well for each component of each fluid of the one or more fluids by summing the mass rate of each component of each fluid of the one or more fluids over total time of the plurality of time steps.

17. The system of claim 10, wherein the surface control system is configured to:
determine a total number of moles of gas released at a surface of the well for each component of each fluid of the one or more fluids by dividing the mass of each component of each fluid of the one or more fluids by molecular weight of the one or more fluids; and
determine a total volume of gas released at the surface of the well for each component of each fluid of the one or more fluids by multiplying a number of moles for each component of each fluid of the one or more fluids by a molecular volume for each component of each fluid of the one or more fluids.

18. The system of claim 17, wherein the surface control system is configured to determine a total volume of gas released at the surface of the well by summing the total volume of gas released at the surface of the well for each component of each fluid of the one or more fluids.

19. A formation testing platform configured to:
receive one or more fluids from a subterranean formation while a formation testing tool is disposed in a wellbore of a well;
determine data relating to one or more properties of the one or more fluids; and
determine hydrocarbon content of the one or more fluids and/or gas emissions relating to the one or more fluids based at least in part on the data relating to the one or more properties of the one or more fluids.

20. The formation testing platform of claim 19, wherein the formation testing platform comprises the formation testing tool and a surface control system configured to collectively control formation testing operations.
